(19) United States
(12) Patent Application Publication
(10) Pub. No.: US 2010/0228292 A1
(43) Pub. Date: Sep. 9, 2010

(54) SYSTEMS AND METHODS FOR DYNAMIC SPINAL STABILIZATION

(75) Inventors: Benjamin Arnold, San Diego, CA (US); Neel Anand, Los Angeles, CA (US); William Taylor, San Diego, CA (US); Rich Mueller, Carlsbad, CA (US); Eric Dasso, Carlsbad, CA (US); G. Bryan Cornwall, San Diego, CA (US); Matthew Copp, San Diego, CA (US)

Correspondence Address: NuVasive;c/o CPA Global, P.O. Box 52050, Minneapolis, MN, 55402, US

(73) Assignee: NUVASIVE, INC., San Diego, CA (US)

(21) Appl. No.: 12/309,722

(22) PCT Filed: Jul. 24, 2007
(86) PCT No.: PCT/US07/16804

§ 371 Date: May 12, 2010

Related U.S. Application Data
(60) Provisional application No. 60/833,236, filed on Jul. 24, 2006.

(51) Int. Cl. A61B 17/70

(52) U.S. Cl. 606/264

(57) ABSTRACT

A dynamic stabilization system including a dynamic rod with a first and second coupler assemblies, a pre-tensioned central cord and a flexible bumper, which are coupled to the bony structures of the spine by way of pedicle screws for use in spinal fixation surgery.

### CROSS-REFERENCE TO RELATED APPLICATIONS

This is an International Patent Application claiming the benefit of priority under 35 U.S.C. §119(e) from the commonly owned and co-pending U.S. Provisional Patent Application Ser. No. 60/833,236, entitled “System and Methods for Dynamic Stabilization” and filed on Jul. 24, 2006, the entire contents of which is expressly incorporated by reference into this disclosure as if set forth in its entirety herein.

### BACKGROUND OF THE INVENTION

I. Field of the Invention

The present invention relates to medical devices generally aimed at spinal surgery and, more particularly, to systems and methods for performing dynamic spinal stabilization.

II. Discussion of the Prior Art

The human spine is comprised of a plurality of components (e.g. vertebral bodies, intervertebral discs, posterior bony structures) which collectively protect the spinal cord and enable the normal physiologic motions of flexion (bending forward), extension (bending backwards), lateral bending (bending side to side), and rotation (twisting). These normal physiologic motions may be impeded and/or pain generating when any of a number of conditions exists, including but not limited to disc degeneration, trauma, and deformity (e.g. scoliosis). Depending upon the condition, surgical intervention may be required to restore the normal physiologic function of the spine at the affected region. One form of surgical intervention involves fusing one or more levels within the spine. This is typically accomplished by performing a discectomy (removing part or all of an intervertebral disc), introducing a height-restoring implant into the disc space, and then immobilizing the adjacent vertebral bodies on either side of the intervertebral implant such that a bony bridge may form between the adjacent vertebral bodies to fuse that particular spinal segment. The step of immobilizing the vertebral bodies may be accomplished in may ways, including the use of pedicle screws (fixed axis or multi-axial) and rigid rods, wherein the pedicle screws are introduced into the pedicles associated with the respective vertebral bodies and the rigid rods are locked to each pedicle screw to prevent motion between the adjacent vertebral bodies.

Although generally effective, fusion procedures do have a number of potential drawbacks. One drawback stems from the fact the pedicle screws are introduced directly into the vertebra. This results in significant forces being loaded on the vertebra, which may ultimately result in the loosening of the pedicle screw. Another potential drawback to fusion is that while fusion generally results in a strengthened portion of the spine at the fusion level, it also results in increased loads being placed on adjacent spinal levels. This in turn may result in increased degeneration, hyper-mobility, and collapse of spinal motion segments adjacent to the fused segment, thereby reducing or even eliminating the ability of the adjacent spinal joints to support normal physiologic motions. A still further drawback stems from fusion itself, in that fusion limits the mobility of the patient and yet may fail to provide adequate pain relief for the patient.

Based on the shortcomings (real or perceived) of fusion, an increasing number of surgeons are performing, or wish to perform, so called “dynamic stabilization” of an affected spinal region. Dynamic stabilization involves coupling adjacent vertebra together using elastic materials and/or shapes capable of allowing the adjacent vertebrae to maintain a level of motion there between while still stabilizing the segment. Dynamic stabilization systems vary in type, including but limited to pedicle-based (using pedicle screws and flexible rods) and interspinous-based (using flexible implants between spinous processes). The general goal of these systems is to create, as much as possible, a more normal loading pattern between the vertebrae in one or more of flexion, extension, compression, distraction, side bending and torsion. For pedicle-based dynamic stabilization, an advantage is the reduction, if not elimination, of pedicle screw loosening found in pedicle-based fusion systems due to the reduction in forces applied to the pedicle screws over time.

One pedicle-based dynamic stabilization system is the Dynesys® system owned and marketed by Zimmer® Spine. The Dynesys system includes pedicle screws with side-loading housings, external spacers made of surgical polyurethane tubing cut intra-operatively to extend between adjacent pedicle screws, and a polyethylene cord that is intra-operatively threaded through the side-loading housing of the pedicle screws and through the polyurethane tubing before being tensioned and locked to the pedicle screws. Once assembled intra-operatively, the polyurethane tubing serves as a compression bumper between the pedicle screws and allows some (but not excessive) extension. The polyethylene cord, on the other hand, serves as a tension band between the pedicle screws and allows some (but not excessive) flexion.

Although generally effective at stabilizing a spinal segment, the Dynesys® system suffers from several significant drawbacks. One drawback is the need to intra-operatively assemble the dynamic aspects of the system, namely, the polyurethane tubing and the polyethylene cord. The polyurethane tubing is cut intra-operatively after the pedicle screws have been implanted and the appropriate size is determined by the surgeon based on the particular needs, anatomy, pathology, etc. . . . of the patient. The polyethylene cord is similarly cut intra-operatively after it has been threaded through the side loading pedicle screws and tensioned. This need to intra-operatively tailor the polyurethane tubing and polyethylene cord consumes precious operative time, which translates into higher costs to the hospital, and increases the risk to the patient due to the longer surgical time.

Another significant drawback to the Dynesys system is the “side-loading” nature of the pedicle screws and the need to thread the polyethylene cord through the side-loading housings and tension the cord intra-operatively during the assembly process. The need to thread the polyethylene cord through the side-loading housing and through the polyurethane tubing, as will be appreciated, increases the difficulty and “fiddle factor” of the system and hence increases the amount of time required to assemble the system. The need to tension the polyethylene cord intra-operatively not only adds time to the procedure, but also introduces variability into the surgery, as different surgeons may choose to tension the device more or less robustly than others. This may affect the outcome of each particular surgery, making some better and some worse, based on the variability in assembly. This cuts against the general surgical goal to provide “safe and reproducible” surgical outcomes.

The present invention is directed at addressing this need and eliminating, or at least reducing, the effects of the shortcomings of the prior art systems as described above.

### SUMMARY OF THE INVENTION

The present invention overcomes the drawbacks of the prior art by providing systems and methods for performing dynamic spinal stabilization which are easy-to-use with dynamic rod assemblies and top-loading pedicle screws (fixed axis and/or multi-axial). The dynamic rod assemblies may be provided sterile and ready for implantation. The dynamic stabilization system is provided, according to one embodiment, comprising a dynamic rod, pedicle screws capable of receiving the dynamic rod, and set screws for securing the dynamic rod to the pedicle screws. When secured to a spine segment, the dynamic rod effects (e.g. limits, resists, prevents, neutralizes) movements not generally occurring in a healthy spine.

According to one embodiment, the dynamic rod comprises a bumper assembly, a tension cord, and a pair of coupler assemblies. The bumper assembly includes a bumper sandwiched between two washers. The bumper may be made from a biocompatible material. In one embodiment the bumper may be composed of a polymer material such as, by way of example only, polycarbonate urethane (“PCU”) or poly(styrene-b-isobutylene-b-styrene) (“SIBS”). If the bumper material is radiolucent, radiopaque markers and/or radiopaque molecules or materials (e.g. Barium Sulphate) may be added to the bumper material so that the entire dynamic rod construct may be viewable under x-ray. The bumper has a bore extending longitudinally therethrough for receiving the tension cord. The tension cord, according to one embodiment, may be formed from a biocompatible elastic, textile, or fabric material, such as by way of example only a polymeric non-absorbable suture. In an untensioned state, the tension cord has a band like structure that is comprised of a number of loops formed with the suture. During assembly, the tension cord may be stretched, braided, woven, twisted, or embroidered into a state of tension. The coupler assemblies may be configured to mate with pedicle screws for attaching the dynamic rod to the vertebrae. The coupler assembly includes a body component and a pin component. The pin locks the tension cord within the body component of the coupler assembly. The body fixes to the bumper assembly at one end and cooperates with the pedicle screw at the other end. The body of the coupler assembly may include an at least partially spherical or bulbous end for engaging with various pedicle screws.

A method of assembling the components of the dynamic rod may be performed, by way of example only, as follows. First, one end of the tension cord is attached to a coupler assembly with a pin. The bumper assembly is then inserted over the free end of the tension cord. Next, a second coupler assembly is attached to the tension cord with another pin. To tension the tension cord, the coupler assemblies are rotated in opposite directions relative to each other. This imparts a series of twists to the tension cord. The twisting of the cord shortens the length and adds tension to the tension cord. As the tension cord length decreases, the coupler assemblies are drawn together with the bumper assembly. Once the desired tension level is reached, twisting is halted, the tension level is verified (optional), and the components are welded together (also optional). The assembled dynamic rod may be packaged, sterilized, and delivered to the operating room ready for implantation such that the surgeon need only retrieve the dynamic rod from the packaging and attach it to the pedicle screws anchored in the patient's spine.

By way of example only, to implant the spinal stabilization system of the present invention, the vertebra to be stabilized are accessed (e.g. via one of an open, mini open, and minimally invasive technique) and pedicle screws are anchored into the vertebrae. Thereafter, the dynamic rod is retrieved and the coupler assemblies are aligned over the pedicle screws to ensure the appropriate sized rod is used. The dynamic rod is reduced into receiving members of the pedicle screws and set screws are secured overtop of the coupler assembly, locking the dynamic rod in position.

A kit may be provided containing a plurality of dynamic rods having various length measurements. The kit may comprise an instrument tray or any number of other suitable packages. By way of example only, the kit may be provided as a simple box filled with individually packaged dynamic rods of various lengths. Significantly, according to one embodiment of the present invention, when providing dynamic rods of various lengths, the modulus of the dynamic rods may be varied so that the stiffness of the dynamic rod will remain the same (or relatively the same) no matter the length of the rod. One exemplary method of effecting the modulus change according to the present invention is to change the Styrene content of the SIBS polymer used to make one embodiment of the bumper.

According to an alternate embodiment of the present invention, a hybrid rod may be provided. The hybrid rod facilitates dynamic stabilization at one level of the spine and fusion or rigid fixation at another level. The rod differs from the dynamic rod previously described in that a rigid rod portion extends from one end of the bumper assembly.

According to yet another alternate embodiment of the present invention, a multi-level dynamic rod may be provided. The multi-level dynamic rod differs from the dynamic rod previously described in that a second bumper assembly is added to the rod. The multi-level rod facilitates dynamic stabilization across multiple spinal levels.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is an exploded view of a single-level dynamic stabilization system, according to one embodiment of the present invention;

FIG. 2 is an exploded view of a single level dynamic rod for use with the dynamic stabilization system of FIG. 1, according to one embodiment of the present invention;

FIG. 3A is a perspective view of a bumper forming part of the dynamic rod of FIG. 2, according to one embodiment of the present invention;

FIG. 3B is a partial cross-sectional view of the bumper of FIG. 3A, illustrating the various diameters associated with a longitudinal bore extending therethough, according to one embodiment of the present invention;

FIG. 4 is a front view of the bumper of FIG. 3A, according to one embodiment of the present invention;

FIG. 5 is a perspective view of a washer component forming a part of the dynamic rod of FIG. 2, according to one embodiment of the present invention;

FIG. 6 is a side view of the washer of FIG. 5, according to one embodiment of the present invention;

FIG. 7 is an exploded perspective view of a bumper assembly forming a part of the dynamic rod of FIG. 2, according to one embodiment of the present invention;

FIG. 8 is a partial cross-sectional exploded perspective view of the bumper assembly of FIG. 7, according to one embodiment of the present invention;

FIG. 9 is a perspective view of the bumper assembly shown FIG. 7 in assembled form, according to one embodiment of the present invention;

FIG. 10 is a partial cross-sectional perspective view of the bumper assembly of FIG. 9, according to one embodiment of the present invention;

FIG. 11 is perspective view of a tension cord forming part of the dynamic rod of FIG. 2, according to one embodiment of the present invention;

FIG. 12 is an enlarged perspective view showing a portion of the tension cord of FIG. 11, according to one embodiment of the present invention;

FIG. 13 is a perspective view showing the body of a coupling assembly forming a part of the dynamic rod of FIG. 2, according to one embodiment of the present invention;

FIG. 14 is a partial cross-sectional view of the coupling assembly body of FIG. 13, according to one embodiment of the present invention;

FIG. 15 is a perspective view of breakaway pin component of a coupling assembly forming part of the dynamic rod of FIG. 2, according to one embodiment of the present invention;

FIG. 16 is a perspective view of the breakaway pin of FIG. 15 with an extension portion broken away, according to one embodiment of the present invention;

FIG. 17A is a top view of the tension cord of FIG. 11 coupled in a non tensioned state to the coupling assembly of FIGS. 13-16, according to one embodiment of the present invention;

FIG. 17B is a cross-sectional view of the tension cord and coupling assembly of FIG. 17A, according to one embodiment of the present invention;

FIGS. 18A-18E are a series of top views illustrating the progression of steps for assembling the dynamic rod of FIG. 2, according to one embodiment of the present invention;

FIG. 19 is a top view of the assembled dynamic rod of FIG. 2 with the bumper portion removed to show the tensioned state of the tension cord, according to one embodiment of the present invention;

FIGS. 20A-20F are a series of side views illustrating the progression of steps for implanting the dynamic stabilization system of FIG. 1, according to one embodiment of the present invention;

FIG. 21 is a top view of a kit for providing the dynamic rod of FIG. 2 with a variety of different length dimensions, according to one embodiment of the present invention;

FIG. 22 is an exploded view of a hybrid rod style dynamic stabilization system, according to another embodiment of the present invention;

FIG. 23A is a perspective view of a rod body forming part of the hybrid rod of FIG. 22, according to one embodiment of the present invention;

FIG. 23B is a partial cross-sectional perspective view of the rod body of FIG. 23A, according to one embodiment of the present invention;

FIG. 24 is a side view illustrating the hybrid style dynamic stabilization system of FIG. 22 in use, according to one embodiment of the present invention;

FIG. 25 is an exploded view of a multi-level dynamic stabilization system, according to another embodiment of the present invention;

FIG. 26A is a perspective view of a connector forming part of the multi-level dynamic rod of FIG. 25, according to one embodiment of the present invention;

FIG. 26B is a partial cross-sectional perspective view of the connector of FIG. 26A, according to one embodiment of the present invention; and

FIG. 27 is a side view illustrating the multi-level dynamic stabilization system of FIG. 25 in use, according to one embodiment of the present invention.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The dynamic stabilization systems disclosed herein boast a variety of inventive features and components that warrant patent protection, both individually and in combination.

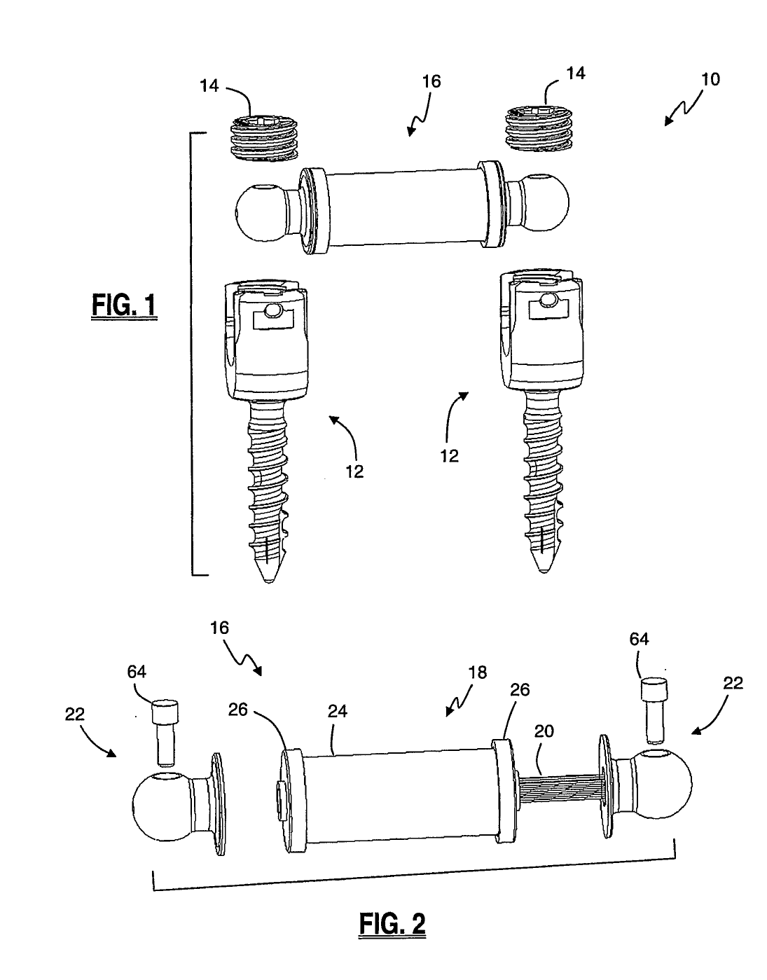

A dynamic stabilization system 10, according to one embodiment of the present invention, is illustrated by way of example only in FIG. 1. The dynamic stabilization system 10 comprises a dynamic rod 16, a pair of pedicle screws 12 capable of receiving the dynamic rod 16, and a pair of set screws 14 for securing the dynamic rod 16 to the pair of pedicle screws 12. Pedicle screws are well known in the art and it will be appreciated that pedicle screws 12 may be multi-axis screws (as shown herein), fixed-axis screws, or a combination of multi-axis and fixed-axis screws. It will also be appreciated that pedicle screws 12 may be replaced by other suitable fastening devices, including, but not necessarily limited to, laminar hooks of multi-axis and/or fixed-axis construction. Once secured, preferably bilaterally, across an unstable spinal segment, the dynamic rod 16 stabilizes the spinal segment while still allowing for at least a modicum of natural physiologic motion.

The dynamic rod 16, an exploded view of which is shown, by way of example only, in FIG. 2, comprises a number of components which may preferably be preassembled and provided sterilized and ready for implantation. The components of dynamic rod 16 preferably include, but are not necessarily limited to, a bumper assembly 18, tension cord 20, and a pair of coupler assemblies 22 which provide for a mating engagement with the pedicle screws 12. When the spinal stabilization system 10 is in use, the dynamic rod 16 neutralizes unnatural movement of the spinal segment during any of flexion, extension, lateral bending, axial rotation or a combination thereof. During physiologic motion the rod is loaded both axially and in bending. Although this motion occurs in combination, a majority of the deformation of the rod is in bending.

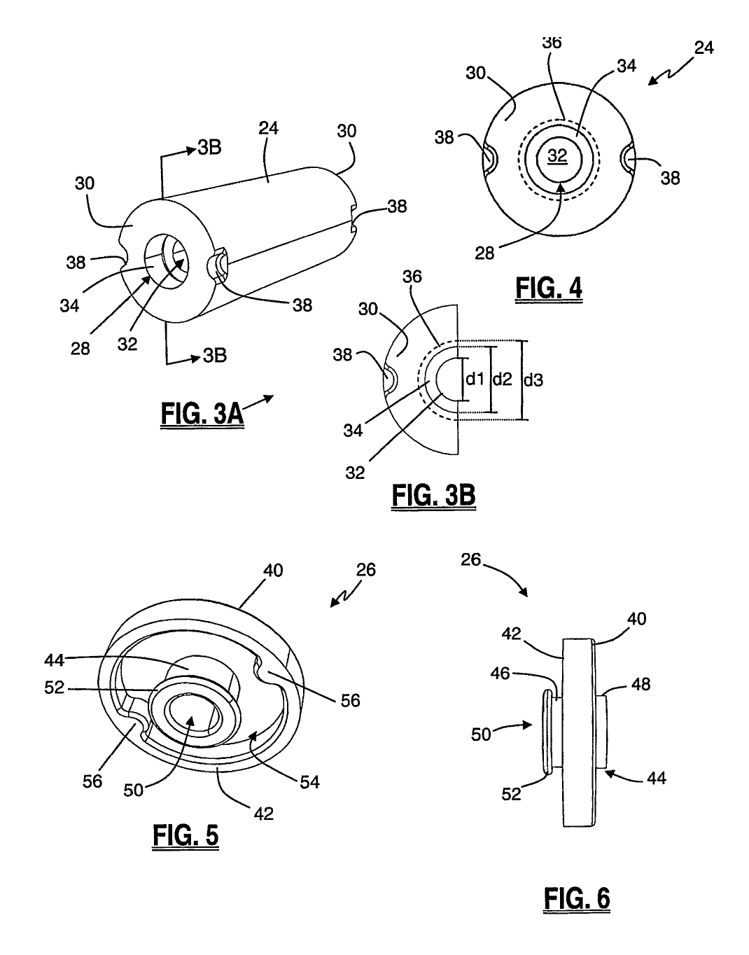

With reference to FIGS. 3-10, there is shown, by way of example only, one embodiment of bumper assembly 18, which includes a flexible bumper 24 sandwiched between a pair of washers 26. Bumper 24 may preferably, though not necessarily, be generally cylindrical in shape. As shown in FIG. 3, the bumper 24 possesses a longitudinal bore 28 extending between the two bumper ends 30. A central portion 32 of the longitudinal bore 28 has a first diameter d1, the diameter d1 being sufficiently large to receive the tension cord 20 therethrough. Adjacent each bumper end 30 the bore 28 widens into end portions 34 having a second diameter d2 that is larger than the diameter d1. In between each end portion 34 and the central portion 32 of bore 28 a groove 36 is formed having a third diameter d3 that is larger than the diameter d2. Cutouts 38 may be disposed along the periphery of bumper ends 30. While shown as generally half circular cutouts, it will be appreciated that cutouts 38 may be provided in any number of suitable shapes, including, but not necessarily limited to, rectangular and triangular cutouts. As will be described below, the bore end portion 34, groove 36, and cutout 38 features provided at each bumper end 30, interface with mating features on the washers 26 to couple and affix the washer 26 to the bumper 24 and thus form bumper assembly 18.

The bumper 24 may be made from any biocompatible material with a stiffness that will allow the bumper 24 to resist but preferably not eliminate motion when it is subject to the bending and compressive loads it will encounter. In one embodiment, the bumper 24 may be composed of a polymer material such as, by way of example only, polycarbonate urethane (“PCU”) or poly(styrene-b-isobutylene-b-styrene) (“SIBS”). In embodiments where the bumper material used is radiolucent (i.e. not visible through x-ray) it is preferred, though not necessary, to add a raidopaque component to the bumper 24. This may be accomplished by positioning small metallic markers in strategic locations along the bumper 24 (not shown). Alternatively, a measure of radiopaqueness may be added to the radiolucent polymer by mixing radioopaque molecules or material into the polymer material. By way of example, a small amount of Barium Sulphate (BaSO₄) may be added to PCU or SIBS prior to forming the bumper 24. Using this method, the bumper 24 will produce a “ghosting” effect under x-ray such that the bumper 24 may be seen but does not obstruct the view adjacent or nearby structures.

The washers 26, which cap the bumper ends 30 to form the bumper assembly 18, are shown in detail in FIGS. 5-6. The washers 26 may be formed from a rigid biocompatible material, including but not necessarily limited to titanium, titanium alloy, and surgical grade steel. Each washer 26 includes an outer surface 40 for mating with a coupler assembly 22, and an inner surface 42 for mating with the bumper 24. A cylindrical wall 44 having a proximal end 46 and a distal end 48 extends from both surfaces 42, 40 of the washer 26 and forms a central bore 50 therethrough. When assembled, the central bore 50 of each washer 26 aligns with the central portion 32 of the bumper longitudinal bore 28 such that the tension cord 20 may pass entirely through the bumper assembly 18. As best viewed in FIG. 6, the distal end 48 of wall 44 extends beyond outer surface 40 and the proximal end 46 extends beyond the inner surface 42. As will be described in more detail below, the extension of distal end 48 helps ensure the proper alignment of bumper assembly 18 with the coupler assemblies 22. The edge of proximal end 46 comprises a flange 52 that cooperates with the groove 36 in bumper 24 to help fix the washers 26 and bumper 24 together. Inner surface 42 has a cavity 54 formed therein which is dimensioned to receive a bumper end 30. Inner surface nodes 56 extend into the cavity 54. The nodes 56 are dimensioned to interface with and engage into cutouts 38 of bumper 24 when the bumper assembly 18 is assembled. This positive engagement prevents rotational movement of bumper 24 relative to the washers 26.

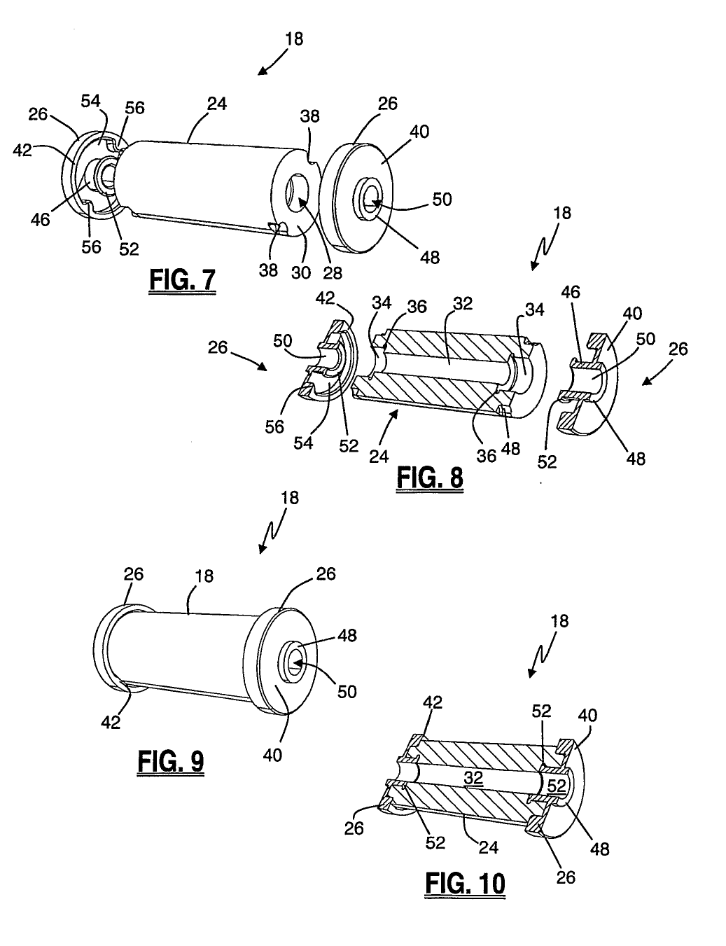

The manner in which bumper 24 and washers 26 cooperate to form bumper assembly 18 is best understood in conjunction with FIGS. 7-10. A washer 26 is positioned on each end 30 of bumper 24. For each washer 26, the proximal end 46 of cylindrical wall 44 is situated within the end portion 34 of the longitudinal bore 28 and the flange 52 is situated within the groove 36. The flange 52 has a diameter roughly equal to the diameter (d3) of groove 36. Since the diameter of the flange 52 is greater than the adjacent diameters d1 and d2 of the central 32 and end 34 portions of the bore 28, respectively, the flange 52 is trapped within groove 36 and the washer 26 and bumper 24 cannot be separated. The external diameter of wall 44 is roughly equal to the diameter, d2, of the bore end portion 34. The washers 26 and bumper 24 thus fit intimately together, thereby limiting any motion between the components. Meanwhile, the nodes 56 of inner surface 42 fit snugly within the cutouts 38 of bumper 24 to further eliminate the possibility of rotational motion between the bumper assembly 18 components. The interior diameter of wall 44 is generally equal to the diameter (d1) of the central portion 32 of bore 28. This provides for a smooth transition between the bore 28 and the bores 50, such that there are no rough surfaces against which the tension cord 20 might rub when the dynamic rod 16 is assembled. According to one method of assembling the bumper assembly 18 (set forth by way of example only), the washers 26 may be spaced apart according to a desired overall bumper assembly 18 length, and thereafter, the bumper material (e.g. PCU, SIBS, etc. . . . ) may be molded between the washers 26.

Turning to FIGS. 11-12, the tension cord 20, according to one example embodiment, is depicted. Tension cord 20 may be formed from a biocompatible elastic, textile, or fabric material. By way of one example only, the cord 20 may be formed from a polymeric non-absorbable suture material. During assembly, the cord 20 may be stretched, braided, woven, twisted, or embroidered into a state of tension. FIG. 11 shows the tension cord 20 in a pre-assembly, non-tensioned state. The cord 20 has a band or hoop like structure with a number of loops 58 laid around an open center 60 (best viewed in FIG. 12). To form tension cord 20 into the non-tensioned state, the suture material is arranged into the desired number of loops 58 and the free ends of suture are fixed together, for example, by tying them together into a knot.

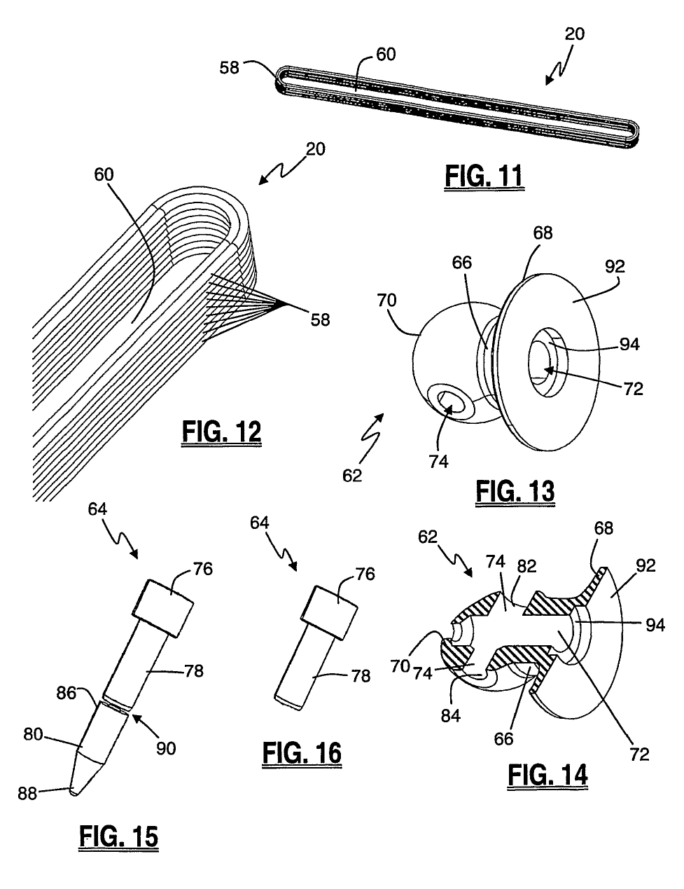

The coupler assembly 22 of dynamic rod 16 is illustrated, according to one embodiment and by way of example only, in FIGS. 13-15. The coupler assembly 22 includes a body 62 and a pin 64. The pin 64 cooperates with the tension cord 20 to fix the cord 20 within the body 62. The body 62 in turn fixes to the bumper assembly 18 at one end and cooperates with the pedicle screw 12 at the other end. The body 62 of coupler assembly 22 may be comprised of a neck 66 situated between a shoulder 68 and a head 70. As pictured, the head 70 may be at least partially spherical or bulbous. The enlarged head 70 may be provided to facilitate use with various pedicle screw systems and/or surgical access systems. By way of example only, the dynamic rod 16 may be used in conjunction with the pedicle screw systems shown and described in commonly owned U.S. patent application Ser. No. 11/031,506, entitled “System and Method for Performing Spinal Fixation,” and filed on Jan. 6, 2005, and Int'l App. No. PCT/US2005/032300, entitled “System and Method for Performing Spinal Fixation,” and filed on Sep. 8, 2004, the entire contents each of which is expressly incorporated by reference into this disclosure as if set forth in their entireties herein. Various attributes and advantages of providing and enlarged head at the end of a rod are described and shown in the referenced applications and it will be appreciated that those attributes and advantages, while not described in detail herein, may apply with equal weight to the dynamic rod 16 of the present invention. It should also be appreciated however, that the enlarged head 70 is not a requirement and the coupler assembly 22 may be modified (if necessary) to cooperate with different pedicle screws such that the dynamic rod 16 of the present invention may be used with any conventional pedicle screw system.

A first channel 72 traverses longitudinally through the body 62 of coupler assembly 22. A second channel 74 traverses the head 70 and intersects the first channel 72. To attach the coupler assembly 22 to the tension cord 20, the tension cord 20 is positioned into the first channel 72 such that a portion of the open center 60 of tension cord 20 is aligned with the second channel 74. The remainder of the tension cord 20 extends out of the body 60 through the shoulder 68. With the tension cord 20 positioned in the first channel 72, the pin 64 is inserted into the second channel 74, passing through the open center 60 and trapping an end of the tension cord 20 within the body 62 (best viewed in FIGS. 17A-17B).

According to one embodiment, set forth by way of example only, the pin 64 comprises a pin head 76, a pin body 78, and an optional breakaway extension 80. As illustrated in the cross-sectional view of FIG. 17B, the pin head 76 is dimensioned to fit snugly within a first opening 82 of the second channel 74. The second opening 84 of the second channel 74 is narrower than the first opening 82 and is dimensioned to snugly receive the pin body 78. Thus, when seated in its final position the pin 64 fully obstructs the openings 82 and 84 and forms a post stretching perpendicularly through the first channel 72 and about which the tension cord 20 is disposed. The pin 64 may be held in position via any of, or a combination of, a weld along the first opening 82, a weld along the second opening 84, a friction fit in the first opening 82, and a friction fit in the second opening 84. With the pin 64 fixed in the second channel 74 through tension cord 20, the tension cord 20 is fixedly associated with the coupler assembly 22 and cannot be removed.

As best shown in FIGS. 15-16, an optional breakaway extension 80 of pin 64 may be utilized to ease the process of inserting the pin body 78 through the central opening 60 of tension cord 20. A trailing end 86 of the breakaway extension 80 is attached via a bridge 90 to the pin body 78. The leading end 88 of extension 80 tapers to a blunt point which is more readily passed through the central opening 60 than the pin body 78 by itself. The trailing end 86 of extension 80 preferably has a diameter generally equal to the pin body 78 such that when the leading end 88 of the extension 80 passes through the central opening 60, the loops 56 of tension cord 20 will follow the taper and the trailing end 86 and pin body 78 may both easily pass through. The bridge 90 connecting the extension 80 to the pin body 78 is preferably constructed such that it is easily snapped or sheared once the pin 64 is in place.

With reference once again to FIGS. 13-14, the shoulder 68 of coupler assembly 22 has an interior face 92 adapted to align and mate with the outer surface 40 of the bumper assembly 18 washer 26. The washer 26 and shoulder 68 may preferably have matching outer diameters so that they come together at a smooth junction. The diameter of the first channel 72 of coupler assembly 22 preferably matches that of the central bore 50 of washer 26 and the central portion 32 of bumper bore 28. This again allows for a smooth transition between the various components such that there are no rough surfaces against which the tension cord 20 might rub when the dynamic rod 16 is assembled.

A cylindrical cutout 94 may be situated in the shoulder face 92 and envelopes the opening of the first channel 72. When the shoulder 68 of coupler assembly 22 and washer 26 of bumper assembly 18 come together, the cutout 94 receives the distal end 48 of the cylindrical wall 44 extending from the outer surface 22 of the washer. Engaging the cylindrical wall 44 with the cutout 94 ensures that the washer 26 and shoulder 68 will be aligned properly.

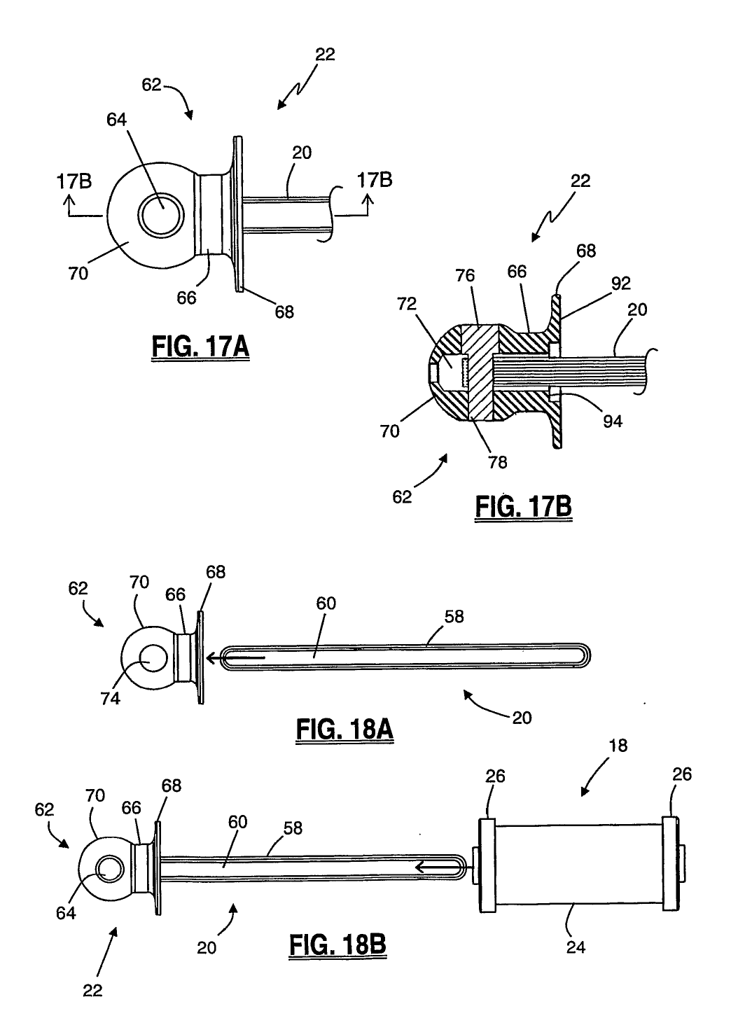

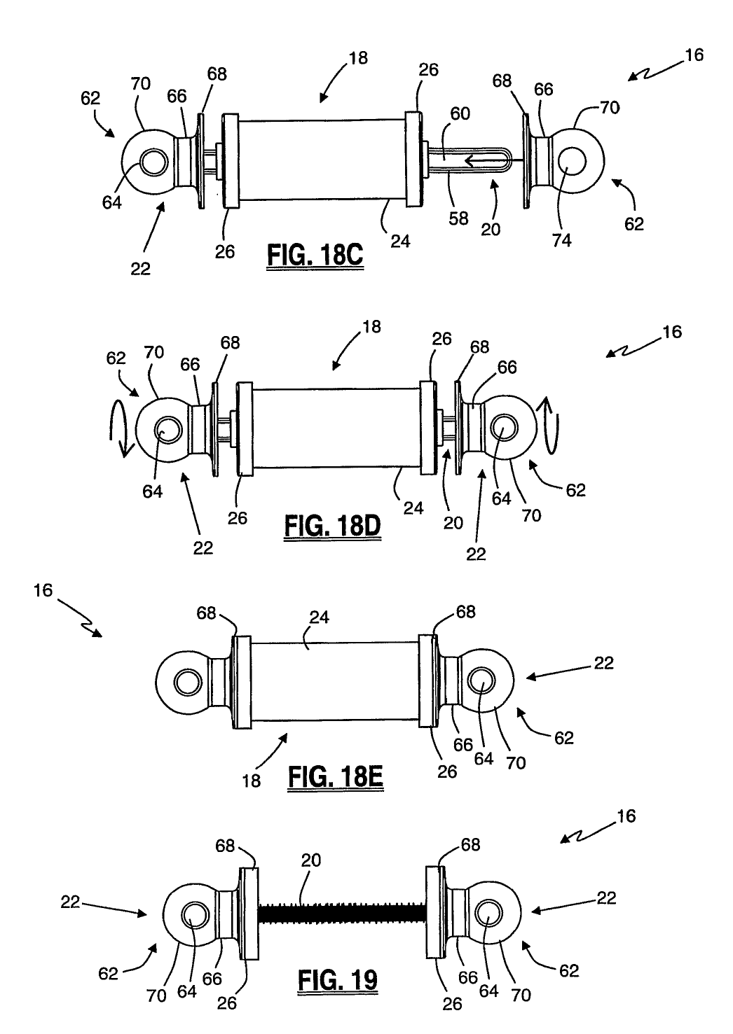

By way of example only, a method of assembling the components of dynamic rod 16 is illustrated in FIGS. 18A-18E. First, one end of the tension cord 20, in its non-tensioned state, is inserted into the first longitudinal channel 72 of a first coupler assembly 22 (FIG. 18A). Once the tension cord 20 is positioned so that the open center 60 is aligned with the second channel 74 of assembly 22, the pin 64 is inserted through the second channel 74 fixing the tension cord 20 to the first coupler assembly (FIG. 18B). Having fixed an end of the tension cord 20 to the first coupler assembly 22, the bumper assembly 18 (having been previously assembled as described above) is inserted over the free end of the tension cord 20 (FIG. 18B). Next, the second coupler assembly 22 is attached to the tension cord 20. Again, the free end of the tension cord 20 is inserted into the first longitudinal channel 72 of the second coupler assembly 22 until the open center 60 of the tension cord 20 is aligned with the second channel 74 of coupler assembly 22 (FIG. 18C). Once the tension cord 20 is aligned within the coupler assembly body 62, the pin 64 is inserted into the second channel 74, thus fixing the second end of the tension cord 20 to the second coupler assembly 22 (FIG. 18D). Having fixed both ends of the tension cord 20 to respective coupler assemblies 22, the bumper assembly is trapped in between the coupler assemblies 22 and all the components of dynamic rod 16 are thus coupled together, albeit in a loose and untensioned state.

To accomplish the tensioning of the tension cord 20, the coupler assemblies 22 are rotated in opposite directions relative to each other (it will of course be appreciated that one coupler assembly 22 may be rotated while the other coupler assembly 22 is still) (FIG. 18D). This imparts a series of twists to the tension cord 20 which shorten the length and add tension to the tension cord 20. As the length of the tension cord is decreased, the coupler assemblies 22 are drawn towards the respective ends of the bumper assembly 28. As the coupler assemblies 22 are drawn towards the ends of bumper assembly 24, the cylindrical wall 44 on each end of the bumper assembly 24 engages the cutout 94 on the respective coupler assembly 22, ensuring the proper alignment to the dynamic rod 16 components. When the shoulder face 92 and washer 26 engage, additional twisting of the coupler assemblies 22 amplifies the tension being added to the tension cord 20 and friction between the shoulder face 92 and the washer 26 prevents the components from coming apart and releasing the tension. When the tension of tension cord 20 reaches a desired level, twisting of the coupler assemblies 22 is halted. At this point the tension level may optionally be verified prior to finishing the assembly with an optional welding of the seams between the coupler assembly shoulders 68 and the washers 26. The assembled dynamic rod 16 (FIG. 18E) may be packaged, sterilized, and delivered to the operating room ready for implantation such that the surgeon need only retrieve the dynamic rod from the packaging and attach it to the pedicle screws 12 anchored in the patient's spine. FIG. 19 illustrates the final tensioned state of the dynamic rod 16 with the bumper 24 removed to show the twisted tension cord 20.

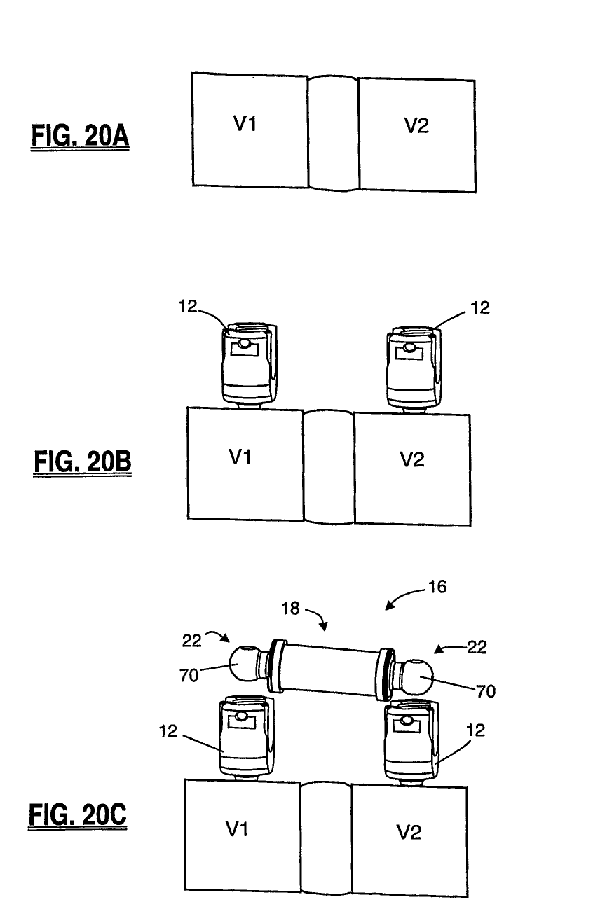

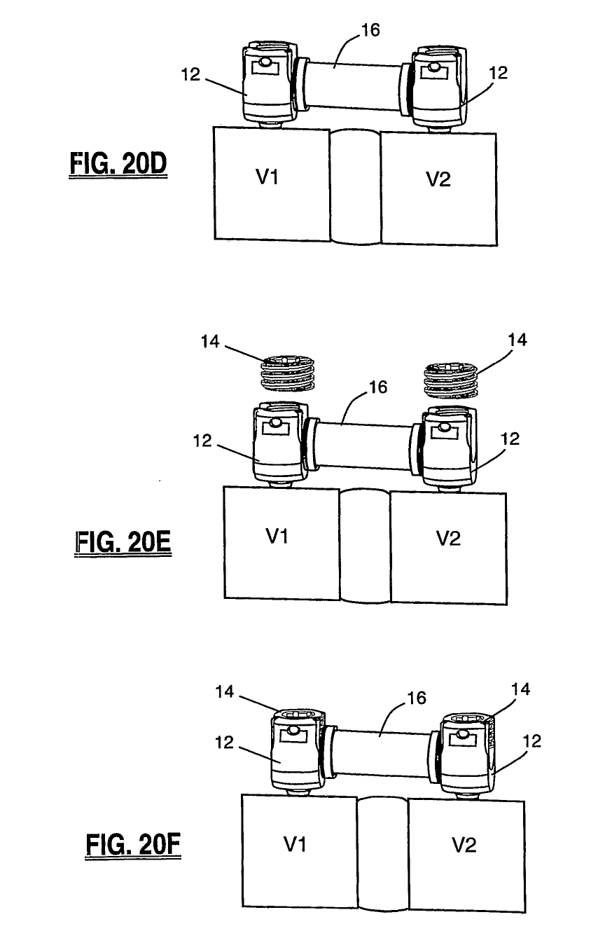

With reference to FIGS. 20A-20F, the step by step progression of implantation of the dynamic stabilization system 10 is depicted (by way of example only). First, the vertebra to be fixed with the dynamic stabilization system 10 (V1 and V2 in the FIGS. 20A-20F) are accessed (e.g. via one of an open, mini open, and minimally invasive technique). Next, the pedicle screws 12 are anchored into the respective vertebra (FIG. 20B). The dynamic rod 16 is retrieved and the coupler assembly heads 70 are aligned over the pedicle screws to ensure the appropriate sized rod is used (FIG. 20C). Thereafter, the dynamic rod 16 is positioned into receiving members of the pedicle screws 12 (FIG. 20D) and set screws 14 are secured overtop of the coupler assembly head 70, locking the dynamic rod 16 in position (FIG. 20).

In a preferred embodiment the dynamic stabilization system 10 will be secured bilaterally on the affected spinal segment(s), and while not shown, it will be appreciated that the implantation method just described may be performed (simultaneously or in succession) on the opposite side of the vertebra as well. Furthermore, it will be appreciated that various instruments and/or instrument systems may be utilized to carry out the general implantation steps described, and use of such instrumentation is contemplated within the scope of this invention. By way of example only, guide tubes, such as those shown and described in the above referenced, Intl App. No. PCT/US2005/032300, may be utilized to access the appropriate vertebrae and to guide the dynamic rod 16 into position.

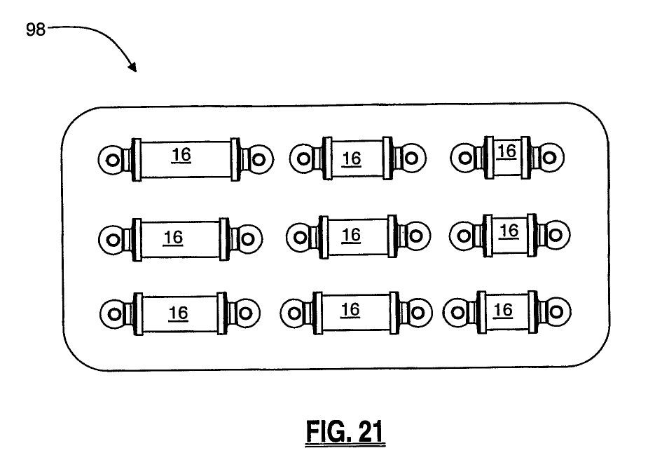

Due to the variety in size of the patient population, it is preferable to provide the dynamic rod 16 of the present invention in a number of different sizes. To accommodate the differing needs of the surgeon based on the anatomy of a particular patient, a plurality of dynamic rods 16 may be provided having various length measurements. FIG. 21 illustrates, by way of example only, a kit 98 comprising multiple dynamic rods 16 of differing lengths. The kit 98 is shown having eight dynamic rods 16 of different lengths; however, any number of rods may be provided. Also, while the kit 98 shown here comprises an instrument tray, any number of suitable packaging methods may be used. By way of example only, the kit 98 may be provided as a simple box filled with individually packaged dynamic rods 16 of various lengths. According to one embodiment, to provide dynamic rods 16 of differing lengths, the lengths of the bumper 24 and tension cord 20 are altered while the dimensions of the remaining components remain the same.

While providing dynamic rods 16 of various lengths is necessary to compensate for the variety in the patient population, doing so may disadvantageously alter certain characteristics of the dynamic rod. For example, altering the length of the bumper 24 while keeping all other parameters the same will alter the stiffness of the bumper 24 construct. While the changing of the stiffness may not necessarily be a disadvantage in and of itself as a large range of stiffness values may be suitable for stabilizing a spinal segment, it may nevertheless be preferable to maintain uniformity of the effective properties across a single product line. Thus, it may be preferable to maintain a constant stiffness (or relatively constant stiffness) over the various dynamic rod lengths provided.

The stiffness of a construct under physiological loading is a function of the modulus of the bumper material at any given length. It stands therefore, that altering the modulus of the bumper material will allow the stiffness of the rod construct to remain relatively uniform regardless of the change in length. The present invention harnesses this principle to provide a plurality of dynamic stabilization rods with the same, or relatively the same, construct stiffness over a variety of rod lengths. Thus, in one embodiment, set forth by way of example only, dynamic rods 16 are produced according to the present invention with varying moduli to provide uniform (or relatively uniform) construct stiffness to all dynamic rods 16 within the kit 98 regardless of the rod length. In one exemplary embodiment, the modulus is altered by changing properties of the polymer material, such as by way of example only, varying the content of a specific material or materials of the polymer. It will be appreciated that any number of different alterations may be made to a polymer to adjust the modulus and therefore accomplish the goal of providing uniform (or nearly uniform) stiffness to the dynamic rods 16 without departing from the scope of the present invention.

To accomplish this goal of providing the dynamic rod 16 having a uniform (or nearly uniform) construct stiffness regardless of length, the bumper 24 may be constructed according to the following formulas regarding axial loading, according to one embodiment of the present invention.

### Given:

$$\begin{matrix}k = \frac{P}{δ} & ( 1 ) \\ σ = \frac{P}{A} & ( 2 ) \\ σ = E \cdot ɛ & ( 3 ) \\ ɛ = \frac{δ}{L} & ( 4 )\end{matrix}$$

Wherein k is the axial stiffness, P is the axial load, δ is the axial displacement, σ is stress, E is Young's modulus, E is strain, L is length, and A is the cross sectional area.
Thus, substituting the definition of stress (2) and strain (4) into Hooke's law (3)

$$\frac{P}{A} = E \cdot \frac{δ}{L} \qquad ( 5 )$$

Finally, by rearranging equation (5) and substituting in the definition of axial stiffness (1)

$$k = E \cdot \frac{L}{A} \qquad ( 6 )$$

Thus, as is evident from equation (6), stiffness is directly proportional to both changes in length and modulus.

From the foregoing discussion it should be appreciated that parameters associated with the dynamic rod 16 of the present invention may vary as specific needs and/or goals to be achieved through any actual implementation arise. By way of example only, dynamic rods 16 may be provided according to the present invention having a length dimension ranging from 15 mm to 60 mm. By way of further example, according to a preferred embodiment, dynamic rods 16 may be provided having length dimensions ranging from 20 mm to 40 mm. The axial stiffness associated with the bumper 24 may range from, by way of example only, 50N/mm to 500N/mm. According to a preferred embodiment, again set forth by way of example only, the axial stiffness associated with the bumper 24 may be in the range of 150N/mm to 350N/mm. The axial tension applied to the tension cord 20 may also be varied and may fall within a range of 50N to 500N. According to a preferred embodiment, the axial tension applied to the tension cord 20 may be in the range of 150N to 350N, as set forth by way of example.

Referring now to FIG. 22, there is depicted a second example embodiment of a dynamic stabilization system 110 according to the present invention. The general configuration and basic components of the dynamic stabilization system 110 are identical the dynamic stabilization system 10 described above. Like numerals have been employed to refer to like parts and additional discussion of the like components have been omitted. The rod 116, depicted by way of example, in FIG. 22 differs from the dynamic rod 16 previously described in that a rigid rod extends from one end of the bumper assembly 18. This may be referred to as a “Hybrid Rod” because it facilitates dynamic stabilization at one level of the spine and fusion or rigid fixation at another level. Use of the hybrid rod 116 when fusion is indicated may prove advantageous for the patient. As previously mentioned, one of the drawbacks of fusion is the increased load that is shifted to spinal segments adjacent to the fused segment which can speed the process of degeneration or cause hyper-mobility, among other things. With the hybrid rod 116 in place, however, the spinal level adjacent to the fusion level is dynamically stabilized, thus decreasing the likelihood of adjacent level disease and/or related negative outcomes.

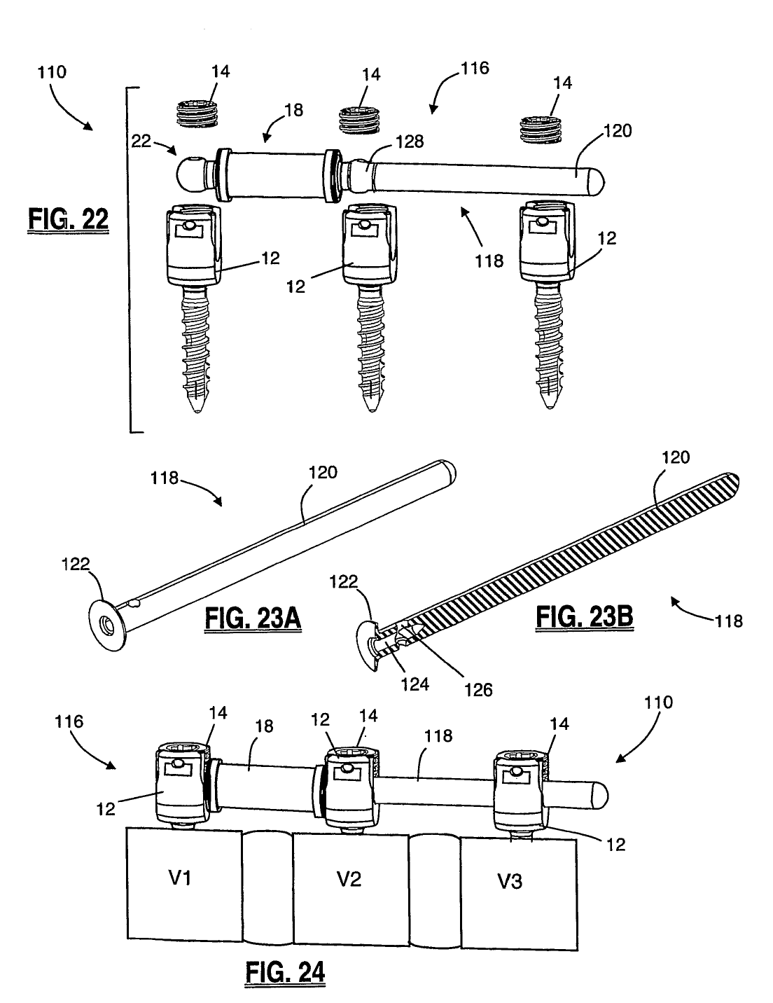

To form the hybrid rod 116, one of the coupler assembly bodies 62 is replaced with a rod body 118, illustrated by way of example only in FIGS. 23A-23B. The rod body 118 comprises an elongated rod 120 and a shoulder 122 which is identical to the shoulder 68 of coupler assembly 22 and engages the washer 26 in the same fashion. A first channel 124 traverses longitudinally through a portion of rod body 118 starting at the shoulder 122. A second channel 126 traverses the elongated rod 120 and intersects the first channel 124 perpendicularly thereto. To assemble the hybrid rod 116, a coupler assembly 22 and the tension cord 20 are fixed together and the bumper assembly 18 is inserted over the tension cord 20. The tension cord 20 is then inserted into the first channel 124 of the rod body 118 until the open center 60 of the tension cord 20 is aligned with the second channel 126. The tension cord 20 is then fixed to the rod body 118 with a pin 64 which is inserted into the second channel 126. Once all the components are coupled together, the tension cord 20 is tensioned via the same twisting method described above. Thereafter, the tension may be verified and the components welded together to finish the assembly. Hybrid rod 116 may be implanted according to the same methods described above utilizing additional pedicle screws 12 for the added levels. When implanted, the bumper assembly 18 spans one spinal level and the elongated rod 120 spans at least one spinal level as pictured in FIG. 24. It will be appreciated that while the elongated rod 120 is illustrated as spanning only one level, the elongated rod 120 portion of hybrid rod 116 may span multiple levels. The elongated rod 120 may include a bulbous 128 (as pictured in FIG. 22) for cooperation with a pedicle screw or (as in FIGS. 23A-23B) the elongated rod 120 may be smooth. The elongated rod 120 may be cut to a desired length and/or bent (pre-bent or intraoperatively bent) to match the natural curvature of the spine if desired.

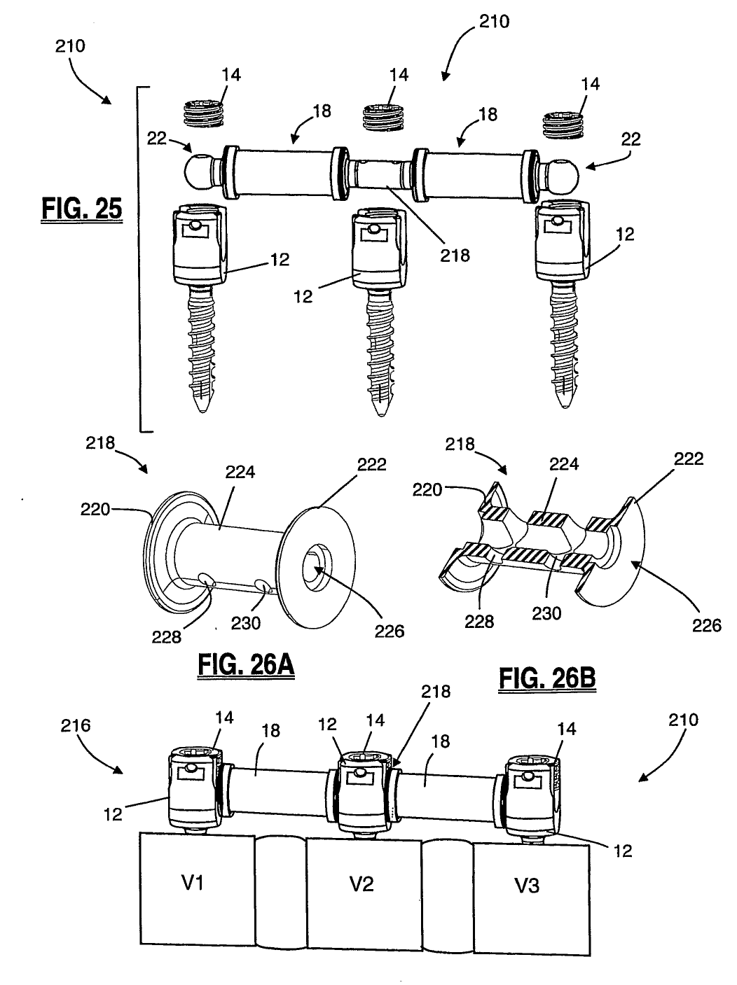

With reference now to FIG. 25, there is shown still another example embodiment of a dynamic stabilization system 210 according to the present invention. The general configuration and basic components of the dynamic stabilization system 210 are identical the dynamic stabilization systems 10 and 110 described above. Like numerals have been employed to refer to like parts and additional discussions of the like components have been omitted. The rod 216 depicted in FIG. 22 differs from the dynamic rod 16 previously described in that a second bumper assembly 24 is added to the rod 216. This rod may be referred to as a “Multi-Level Dynamic Rod” because it facilitates dynamic stabilization across multiple spinal levels.

To form the multi-level dynamic rod 216, one of the coupler assembly bodies 62 on each of two dynamic rods 16 is replaced with a single connector 218. The connector 218 links the two dynamic rods 16 together to form the multi-level dynamic rod 218. The connector 218 is illustrated by way of example only in FIGS. 26A-26B. The connector 218 comprises a first shoulder 220 and a second shoulder 222. The shoulders 220 and 222 are identical to the shoulder 68 of coupler assembly 22 and each engage a washer 26 of bumper assembly 18 in the same fashion as that described above for shoulders 68. The shoulders 220 and 222 are connected by a neck 224. A first channel 226 traverses longitudinally through the connector 218. Proximate to the first shoulder 220, a second channel 228 traverses the neck 224 and intersects the first channel 226 perpendicularly thereto. Proximate to the second shoulder 222, a third channel 230 traverses the neck 224 and intersects the first channel 226 perpendicularly thereto.

To assemble the multi level dynamic rod 216, a coupler assembly 22 and a first tension cord 20 are fixed together and a bumper assembly 18 is inserted over the tension cord 20. The tension cord 20 is then inserted into the first channel 226 through the first shoulder 220 of connector 218. A pin 64 is then inserted through the second channel 228 to fix the tension cord 20 to the connector 218. Next, a second tension cord 20 is inserted into the first channel 226 through the second shoulder 222 of connector 218. A pin 64 is then inserted through the third channel 230 to lock the second tension cord 20 in place within the connector 218. A second bumper assembly 218 is inserted over the second tension cord 20. The second tension cord 20 is then inserted into and fixed to the final coupler assembly 22.

Once all the components of the multi-level dynamic rod 216 are coupled together, the first tension cord 20 is tensioned by twisting the first coupler assembly 22 relative to the connector 218. After tensioning of the first tension cord 20 is complete, the second tension cord 20 is tensioned by twisting the second coupler assembly 22 relative to the connector 218. Thereafter, the tension imparted on the tension cords 20 may be verified and the components welded together to finish the assembly. Multi-level rod 216 may be implanted according to the same methods described above for dynamic rod 16 with additional pedicle screws 12 being utilized for the additional level, as shown in FIG. 27.

A further embodiment which is contemplated but not shown comprises a multi-level hybrid rod. The multi-level hybrid rod comprises at least two bumper assemblies as well as an elongated rod portion. The multi-level hybrid rod may be assembled in the same manner as the multi-level dynamic rod. The final coupler assembly 22 may be replaced by the rod body 118.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the invention is to cover all modifications, equivalents, and alternatives falling within the spirit and scope of the invention as defined herein.

What is claimed is:

1. A system for dynamic stabilization of a spine, comprising:
at least two anchors dimensioned to be attached to said spine; and
a rod dimensioned to be positioned between said at least two anchors, said rod having a tensioned cord situated entirely within an interior space of said rod.

2. The system of claim 1, wherein said interior space is defined by a bumper and a pair of couplers.

3. The system of claim 2, wherein one of said couplers mates with a first end of said bumper and said remaining coupler mates with a second end of said bumper.

4. The system of claim 3, wherein one of said couplers is configured to mate with at least one of said anchors and the remaining coupler is also configured to mate with at least one of said anchors and wherein at least one of said anchors is a pedicle screw and at least one of said couplers is dimensioned to be received within a housing of said pedicle screw.

5. (canceled)

6. The system of claim 4, wherein at least one of said couplers comprises an at least partially spherical shaped end.

7. The system of claim 4, further comprising at least one locking element for fixing at least one of said coupling elements within said housing.

8. The system of claim 7, wherein said at least one locking element is a set screw.

9. The system of claim 3, wherein said tensioned cord is attached to each of said couplers.

10. The system of claim 9, wherein at least one of said couplers is at least temporarily rotatable with respect to the other coupler to change the tension imparted on said tensioned cord.

11. The system of claim 1, wherein said rod is flexible, comprised of an elastic polymer and at least one of bendable during at least one of flexion and extension, compressible during extension, axially extendable during flexion, wherein said tensioned cord is at least one of twisted and made of suture, and wherein said bumper is at least partially radiopaque.

12-18. (canceled)

19. The system of claim 2, wherein one of said first coupler and said second coupler comprises a rigid elongation extending in a direction opposite to said other coupler, wherein said rod is dimensioned to span at least one disc space and said rigid elongation is dimensioned to span at least one additional disc space.

20. (canceled)

21. The system of claim 2, further comprising a second rod, said second rod having a second tensioned cord situated within an interior space of said second rod, where said second rod is connected to said first rod, said interior space of said second rod being defined by a bumper and a pair of couplers, and wherein said first rod and said second rod are both dimensioned to span at least one disc space.

22. (canceled)

23. The system of claim 21, wherein said first rod and said second rod share a coupler.

24-36. (canceled)

37. A kit for stabilizing a spine, comprising:
a plurality of spinal rods of different lengths, wherein the modulus of at least some of said spinal rods has been adjusted such that the difference in stiffness between the rods is less than if the modulus had not been adjusted.

38. The kit of claim 37, wherein at least a portion of each of said plurality of spinal rods comprises an elastic polymer.

39. The kit of claim 38, wherein the modulus of the polymer is adjusted by altering the content of a specific polymer component.

40. The kit of claim 39, wherein said polymer is poly(styrene-b-isobutylene-b-styrene) and the modulus is adjusted by altering the styrene content of the polymer.

41-48. (canceled)

49. The kit of claim 39, wherein at least some of said spinal rods are manufactured with a predetermined stiffness calculated via the formula
$$k = E \cdot \frac{L}{A} ,$$
wherein k is the axial stiffness of said rod, E is Young's modulus of said rod, L is length of said rod, and A is the cross sectional area of said rod.

50. The method of claim 49, wherein the length L of said rod is in the range of 15 mm to 60 mm, the stiffness k of said rod is in the range of 50 N/mm to 550 N/mm, said rod includes an internal cord having a tension in the range of 50N to 500 N.

51. The method of claim 50, wherein the stiffness k of said rod is in the range of 150 N/mm to 350 N/mm and said internal cord has a tension in the range of 50N to 500 N.
